(19) United States
(12) Patent Application Publication
(10) Pub. No.: US 2005/0261625 A1
(43) Pub. Date: Nov. 24, 2005

(54) Nozzle tip for use with syringe and method for using same

(76) Inventor: Arthur Ashman, New York, NY (US)

Correspondence Address: DARBY & DARBY P.C., P. O. BOX 5257, NEW YORK, NY, 10150-5257, US

(21) Appl. No.: 10/630,549

(22) Filed: Jul. 29, 2003

Related U.S. Application Data
(62) Division of application No. 10/132,793, filed on Apr. 24, 2002.
Application No. 10/132,793 is a continuation of application No. 09/086,604, filed on May 29, 1998, now Pat. No. 6,554,803.
Application No. 09/086,604 is a division of application No. 08/831,914, filed on Apr. 2, 1997, now abandoned.

(51) Int. Cl. A61M 37/00

(52) U.S. Cl. 604/82; 606/93; 604/190

(57) ABSTRACT

An improved assembly of a syringe and a nozzle tip and method of use. The syringe includes nozzle tip having a sleeve portion that fits on the free end of the syringe barrel and a curved nozzle end. The nozzle tip may include a filter screen of preselected mesh size. In use, a preselected amount of marrow blood may be aspirated through the curved aspirating nozzle end and filter screen into the syringe barrel where it mixes with bone regeneration material therein to form a viscous fluid mixture. The nozzle tip is removed from the syringe and the mixture is expelled from the syringe and applied to a surgical site by depressing the syringe plunger.

This is a divisional application of U.S. application Ser. No. 10/132,793 filed Apr. 24, 2002, now pending, which is a continuation of U.S. application Ser. No. 09/086,604, filed May 29, 1998, now U.S. Pat. No. 6,554,803, which is a division of U.S. application Ser. No. 08/831,914, filed Apr. 2, 1997, now pending. Each of these prior applications is incorporated by reference herein in its entirety.

### BACKGROUND OF THE INVENTION

The invention relates to a nozzle tip of special construction mounted on the barrel of a standard syringe for dispensing bone regeneration materials to a surgical site. The nozzle tip and syringe are used to aspirate marrow blood from a surgical site; then mixing the collected blood marrow with granular bone regeneration material stored in the barrel of the syringe to form a viscous fluid mixture therein; then manually removing the nozzle tip from the syringe barrel; and then dispensing the viscous fluid mixture to the surgical site by manual application of pressure on the plunger of the syringe. Bone regeneration materials are known in the art. For example, hard-tissue implant materials are know, such as the calcified microporous copolymer bone regeneration material marketed under the trademarks Bioplant® HTR® Synthetic Bone™ alloplast. The aforesaid bone regeneration material has been widely accepted in medicine, dentistry and veterinary medicine as a prosthetic bone material to repair injured or diseased bone. The following co-invented U.S. patents describe the use of such bone generation materials: U.S. Pat. Nos. 4,199,864; 4,244,689; 4,535,485; 4,536,158; 4,547,327; 4,547,390 and 4,728,570. The aforelisted co-invented U.S. patents are incorporated by reference herein. For many applications of said Bioplant® HTR® bone regeneration material the application of this material in granular form has proven to have many advantages. For example, granular Bioplant® HTR® bone regeneration material has proven particularly useful in a tooth extraction procedure. A simple injection of granular Bioplant® HTR® bone regeneration material into the tooth socket, following immediately after extraction of the tooth, either significantly reduces or completely prevents the usual 40% to 60% percent bone loss that otherwise occurs within 2-3 years after tooth extraction, and eliminates much of the pain and inflammation of the tooth socket (post-extraction alveolar osteitis). Granular Bioplant® HTR® bone regeneration material works best when it is thoroughly wetted with marrow blood before being applied to a surgical site.

### SUMMARY OF THE INVENTION

It is an object of the present invention to provide a simple improved syringe and nozzle tip construction for producing and then dispensing a viscous mixture of granular Bioplant® HTR® bone regeneration material and marrow blood, obtained from a surgical site.

It is another object of the invention to provide a simple method of mixing aspirated marrow blood from a surgical site with granular bone regeneration material inside the barrel of a syringe and, by the use of an improved nozzle tip construction, mounted on a standard syringe, prevent excessive loss of marrow blood and/or granular bone regeneration material during the mixing operation.

It is another object of this invention to provide an aseptic method for mixing aspirated marrow blood from a surgical site with granular bone regeneration material and then applying, in an aseptic manner, the viscous mixture obtained by the mixing in the sterile syringe barrel to the surgical site.

Low density polyethylene has been found to be particularly advantageous for manufacturing the entire nozzle tip construction including the filter screen which is mounted inside the nozzle tip. The openings of the mesh screen must be smaller than the grain size of the granular bone regeneration material inside the syringe barrel. A mesh opening size of about 105 microns has been found to work best with the method of the invention because it can be used with several standard granular sized of Bioplant® HTR® bone regeneration materials.

Further details regarding the nozzle tip construction and the method of forming a viscous mixture of granular Bioplant® HTR® polymer material and then applying it to a surgical site will be provided in the following description of various embodiments in conjunction with the accompanying drawings.

### BRIEF DESCRIPTION OF THE DRAWINGS

FIG. 1 is a cross-sectional view of the nozzle tip of the invention;

FIG. 2 is a cross-sectional view of a standard straight barrel syringe holding bone regeneration material, with a nozzle tip of the invention mounted thereon, which standard type of syringe is commonly used in applying bone regeneration materials to a surgical site;

FIG. 3 is a cross-sectional view of the straight barrel syringe of FIG. 2 with the nozzle tip of FIG. 2 mounted thereon during the step of aspirating marrow blood from a tooth socket and mixing it with bone regeneration material in the syringe.

FIG. 4 is a cross-sectional view of the straight barrel syringe of FIG. 2 after the mixing step has been completed and the nozzle tip has been manually removed so that viscous mass formed by the mixture of marrow blood and bone regeneration material is ready to be applied to a surgical site;

FIG. 5 is a view in perspective showing the step of aspirating marrow blood from a tooth socket with the nozzle tip construction of the invention; and

FIG. 6 is a view in perspective showing the step of applying the viscous mixture in the syringe barrel to the tooth socket.

### DESCRIPTION OF THE PREFERRED EMBODIMENTS

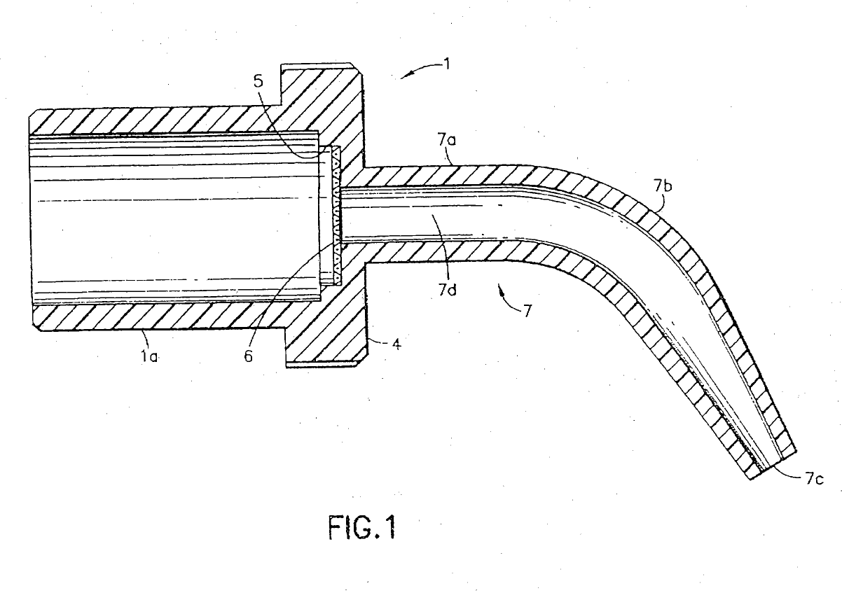

Although the principles of this invention are applicable to other surgical procedures than a tooth extraction, the invention will be fully understood from an explanation of its application to embodiments of syringe and special nozzle tip constructions as illustrated in FIGS. 1-6.

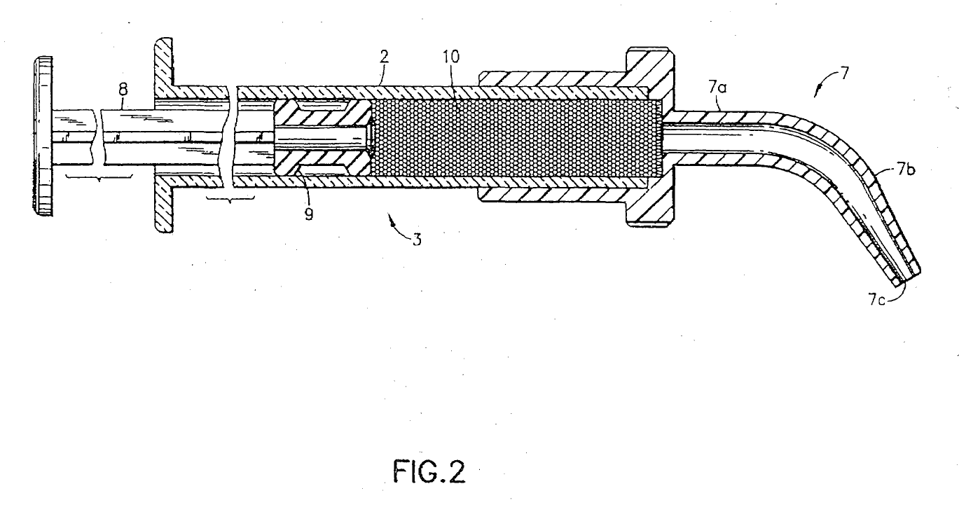

Shown in FIG. 1 is a cross-sectional view of the nozzle tip 1 of this invention. The nozzle tip 1 includes a sleeve portion 1a which has an internal diameter which corresponds to the external diameter of the barrel 2 of the syringe 3 illustrated in FIG. 2. The nozzle tip 1 is therefore mounted on the syringe barrel 2 by means of friction fit. The syringe 3 is of the type that is commonly used for dispensing granular bone regeneration material, such as Bioplant® HTR® bone regeneration material. The nozzle tip has a flange 4 which has a recess 5. A screen 6 having a mesh size of about 105 microns is mounted inside the recess 5. The nozzle tip further has a neck portion 7 with a passage 7d extending therethrough. The neck portion 7 includes an axially straight portion 7a extending from the flange 4 and integral therewith, and a curved portion 7b through the opening 7c thereof the marrow blood can aspirated. The neck portion 7 is integral with the flange 4 and the entire nozzle tip construction including the neck portion 7 and screen 6 are preferably made by a known molding operation of low density polyethylene.

FIG. 2 illustrates in cross-section a syringe 3 with the nozzle tip 1 mounted thereon. The barrel 2 of the syringe 3 is filled with a granular bone generation material 10, such as Bioplant® HTR® bone regeneration material. This barrel is made of either glass or transparent plastic material. The syringe 3 further has the standard plunger 8 on the front end of which is mounted a piston 9. By applying manual pressure to the plunger 8 the piston 9 can be reciprocally slidably axially moved inside the barrel 2 of the syringe 3. The entire assembly, as illustrated in FIG. 2, is mounted inside a non-illustrated conventional blister pack, in which it is distributed to the dentist, surgeon or veterinary practitioner for application of the bone regeneration material to a surgical site. This entire assembly is intended for a single use only and the assembly and blister pack is intended to be discarded after this single use.

FIGS. 3 and 5 illustrate the aspirating step of the invention using the nozzle tip 1 and syringe 3 of the invention. The curved portion 7b of the nozzle tip 1 is inserted, by way of example, by the dentist into the tooth socket 11a of a jaw bone 11b of a patient immediately after a non-illustrated tooth has been extracted from the tooth socket 11a. Marrow blood 11 is then aspirated through the opening 7c of the neck portion 7 by manually retracting the plunger 8. The aspirated marrow blood 11 flows through neck portion 7 and the screen 6 into the barrel 2 of the syringe where it immediately begins to soak the bone regeneration material 10 with marrow blood 11. By visually examining the syringe 3 the dentist or surgeon determines when a sufficient marrow blood 11 has been aspirated from the tooth socket 11a and has mixed with the bone regeneration material 10. If an insufficient amount of marrow blood has been aspirated the aforedescribed steps are repeated. If excess marrow blood has been aspirated this excess marrow blood is expelled by slightly manually moving the plunger forward. While these steps are carried out the screen 6 prevents the clogging with granular bone regeneration material of the passage 7d in the straight neck portion 7a of the neck portion 7.

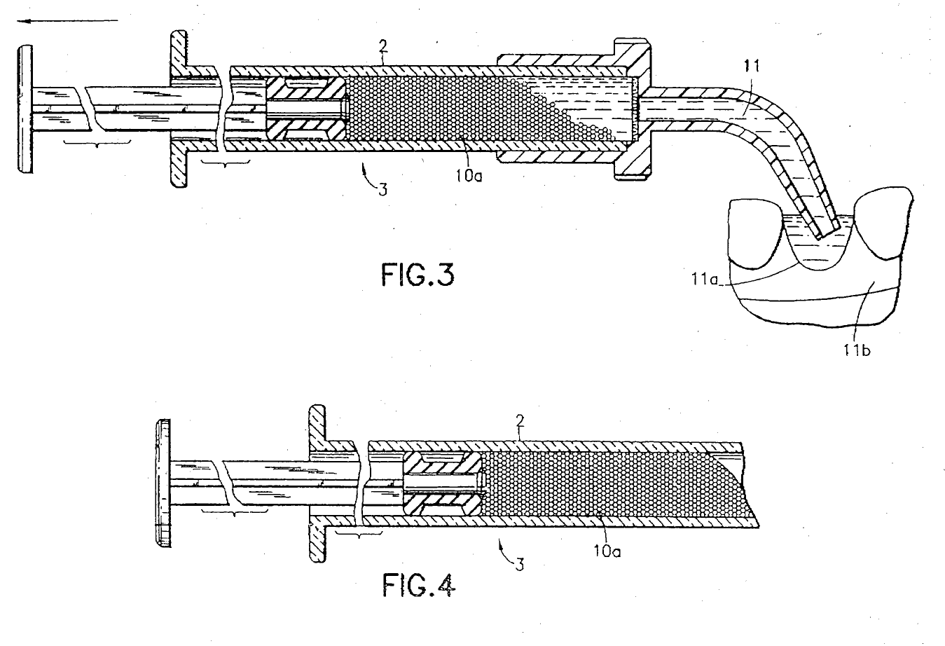

By visually examining the mixture of marrow blood and bone regeneration material inside the syringe barrel 2, the dentist can determine when the mixture 10a of bone regeneration material and marrow blood 11 contains a sufficient amount of marrow blood and thereby the mixture has become sufficiently viscous to be applied to a surgical site. The nozzle tip 1 is then manually slid off the syringe barrel 2 as is shown in FIG. 4.

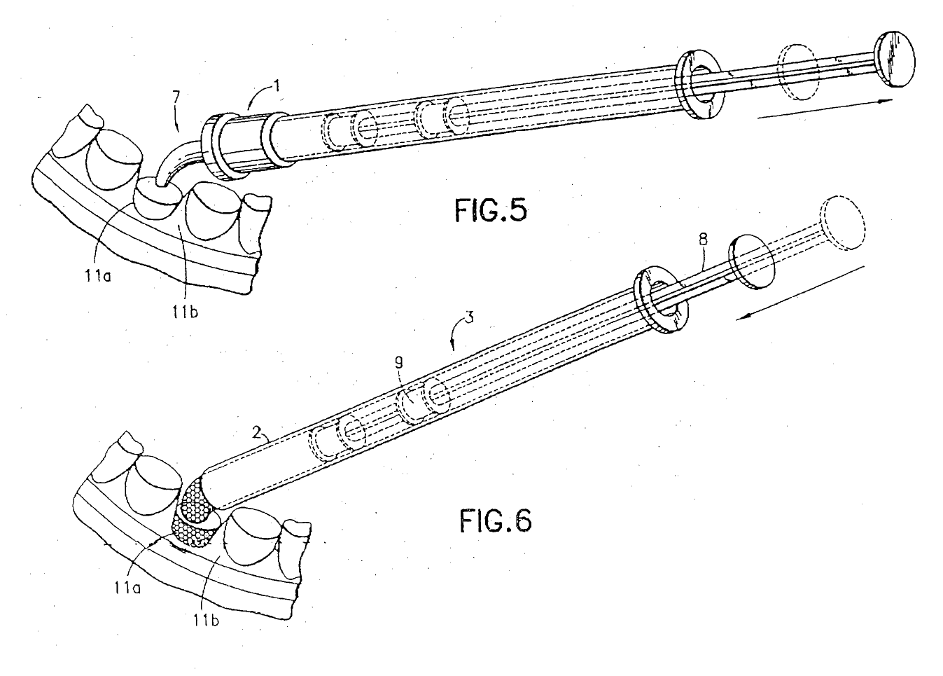

As is shown in FIG. 6 the viscous mixture 10a is then applied to a surgical site, such as a tooth socket 11a, by applying manual pressure to the plunger 8. Once this step has been completed the surgeon may apply sutures to the surgical site if the surgical condition of the patient warrants such a step.

Although the nozzle tip construction and method of applying a viscous mass of a mixture of marrow blood and bone regeneration material of the present invention have been described in terms of the presently illustrated embodiments, it is to be understood that such disclosure is not to be interpreted as limiting. For example, it should be noted that the syringe assembly and method of the invention can be used in other surgical procedures than tooth extraction and can find application in surgery and veterinary medicine. Accordingly, it is intended that the appended claims be interpreted as covering all alterations and modifications as fall within the true spirit and scope of the invention.

1. A syringe and nozzle tip assembly, comprising:
a syringe having a syringe barrel with a front end and a rear end, a piston slidably mounted in the syringe barrel, and a plunger connected to the piston and extending rearwardly through the rear end of the syringe barrel; and
a nozzle tip having a flange, a sleeve frictionally mounted on the front end of the syringe barrel, and a neck with a curved portion and a passage therethrough, wherein the flange includes a recess disposed therein and a filter mounted in the recess.

2. The syringe and nozzle tip assembly of claim 1, wherein the filter comprises a screen and the screen and curved neck portion are integral with the nozzle tip.

3. The syringe and nozzle tip assembly of claim 2, wherein the screen has a mesh size of about 105 microns.

4. The syringe and nozzle tip assembly of claim 1, wherein the outer surface of the syringe barrel is substantially smooth.

5. The syringe and nozzle tip assembly of claim 1, wherein the recess and filter are configured so that the filter does not contact the end of the syringe barrel.

6. The syringe and nozzle tip assembly of claim 1, wherein the syringe barrel is transparent.

7. The syringe and nozzle tip assembly of claim 1, wherein the syringe barrel is made of a material selected from the group consisting of glass and plastic.

8. The syringe and nozzle tip assembly of claim 1, wherein the nozzle tip and filter are made of low density polyethylene.

9. The syringe and nozzle tip assembly of claim 1, wherein the nozzle tip is adapted to retain bone regeneration material in the syringe barrel.

10. The syringe and nozzle tip assembly of claim 1, wherein the flange includes a surface adapted to seat against the syringe barrel when mounted thereon.

11. The syringe and nozzle tip assembly of claim 1, wherein the nozzle tip is mounted solely by friction fit.

12. A syringe and nozzle tip assembly, comprising:
a syringe; and
a nozzle tip frictionally mounted on an end of the syringe, the nozzle tip comprising a sleeve, a flange having a surface adapted to seat against the end of the syringe when the nozzle tip is mounted thereon and a recess disposed therein, a filter mounted in the recess, and a neck having a curved portion and a passage extending therethrough.

13. The syringe and nozzle tip assembly of claim 12, wherein the nozzle tip is mounted solely by friction fit.

14. The syringe and nozzle tip assembly of claim 12, wherein the filter is removable.

15. A syringe and nozzle tip assembly, comprising:
a syringe; and
a nozzle tip mounted on an end of the syringe, the nozzle tip comprising a sleeve, a flange having a surface adapted to seat against the end of the syringe when the nozzle tip is mounted thereon and a recess disposed therein, a filter mounted in the recess, and a neck having a curved portion and a passage extending therethrough, wherein the recess and filter are configured so that the filter does not contact the end of the syringe.

16. The syringe and nozzle tip assembly of claim 15, wherein the filter is removable.

17. The syringe and nozzle tip assembly of claim 15, wherein the nozzle tip is mounted frictionally.

18. The syringe and nozzle tip assembly of claim 17, wherein the nozzle tip is mounted solely by friction fit.

19. A method of using the syringe and nozzle tip assembly of claim 1, comprising:
providing an amount of granular bone regeneration material in the syringe barrel;
aspirating an amount of marrow blood from a surgical site in a patient through the nozzle tip and into syringe barrel;
mixing the aspirated marrow blood with the bone regeneration material in the syringe barrel until an amount of viscous mass of a mixture of bone regeneration material and marrow blood has formed therein;
removing the nozzle tip from the front end of the syringe barrel; and
applying an amount of the viscous mixture to the surgical site.

20. The method of claim 19, further comprising expelling excess marrow blood in the syringe barrel through the nozzle tip prior to removing the nozzle tip.

21. A method of using the syringe and nozzle tip assembly of claim 12, comprising:
providing an amount of granular bone regeneration material in the syringe;
aspirating an amount of marrow blood from a surgical site in a patient through the nozzle tip and into syringe;
mixing the aspirated marrow blood with the bone regeneration material in the syringe until an amount of viscous mass of a mixture of bone regeneration material and marrow blood has formed therein;
removing the nozzle tip from the syringe; and
applying an amount of the viscous mixture to the surgical site.

22. The method of claim 21, further comprising expelling excess marrow blood in the syringe through the nozzle tip prior to removing the nozzle tip.

23. A method of using the syringe and nozzle tip assembly of claim 15, comprising:
providing an amount of granular bone regeneration material in the syringe;
aspirating an amount of marrow blood from a surgical site in a patient through the nozzle tip and into syringe;
mixing the aspirated marrow blood with the bone regeneration material in the syringe until an amount of viscous mass of a mixture of bone regeneration material and marrow blood has formed therein;
removing the nozzle tip from the syringe; and
applying an amount of the viscous mixture to the surgical site.

24. The method of claim 21, further comprising expelling excess marrow blood in the syringe through the nozzle tip prior to removing the nozzle tip.
